(19) United States
(12) Patent Application Publication
(10) Pub. No.: US 2004/0235144 A1
(43) Pub. Date: Nov. 25, 2004

(54) Device for characterising discrete diffusion of a product in one or several corporeal cavities

(76) Inventors: Philippe Laurent, Oullins (FR); Lionel Vedrine, Ridgewood, NJ (US); Marc Durand, Chadrac (FR)

Correspondence Address: David W Highet, Becton Dickinson and Company, 1 Becton Drive, Franklin Lakes, NJ, 07417, US

(21) Appl. No.: 10/470,188

(22) PCT Filed: Jan. 21, 2002
(86) PCT No.: PCT/FR02/00232

(30) Foreign Application Priority Data
Jan. 24, 2001 (FR) 01/00952

(51) Int. Cl. C12M 1/34; A61B 5/02

(52) U.S. Cl. 435/287.1; 600/500

(57) ABSTRACT

This device (10) comprises:

an anatomical portion (1) of an observed part of a dead subject, comprising at least in part the body cavity or cavities (53), and comprising a natural orifice (50) with which at least one of said body cavities communicates; this anatomical portion (1) is obtained by at least one section (52, 51) through said observed part and is treated for its preservation, for example by a technique called “plastination”;

at least one sensor (2 to 6) which can detect and/or quantify said product at at least one site or zone of the body cavity or cavities;

at least one closure piece (12) placed against said anatomical portion (1) in the area of each sectioned surface, in such a way as to close each body cavity (53) or part of the body cavity, but to leave access to said natural orifice (50), and

if appropriate, an introduction device (30) placed opposite said natural orifice, or engaged partially in this orifice, and able to introduce said product in accordance with predetermined parameters.

The present invention relates to a device for characterizing the diffusion and/or distribution of at least one product or substance of interest in discrete form, liquid or solid, in one or more body cavities, in particular a nasal cavity and/or a sinus.

Products in discrete form, in particular aerosols, are commonly used to treat conditions affecting the ENT area.

Studies have been conducted in the past to determine which zones of a nasal cavity or of a sinus receive a product in aerosol form which has been injected through a nostril. However, these studies, carried out using synthetic anatomical models which as far as possible reproduce the anatomy of a nasal cavity or of a sinus, have only given rather imprecise and rather unreliable results which it has not been possible to exploit in a systematic manner.

It has also been contemplated to use, on living subjects or patients, techniques for optically tracing the diffusion of a product, either by scintigraphy or directly by sight. These techniques both have significant limitations as regards the possibility of exploiting the results obtained.

The present invention aims to overcome these important shortcomings by making available a device which permits precise and reliable study of the zones or sites of a body cavity which can be effectively reached by a product of interest, in particular in discrete form, this product being introduced, for example inhaled, from an orifice connected to this cavity, in accordance with defined introduction parameters; these introduction parameters relate in particular to the shape and dimensions of the introduction device, to the shape of the jet of product, to the position and orientation of this introduction device in relation to the natural orifice communicating directly or indirectly with the cavity being studied, to the dimensions of the particles of the liquid or solid product, to the introduction pressure or to the physicochemical characteristics of the product.

It is a particular object of the invention to evaluate the diffusion of the product in the ENT area of a subject, when this product is injected into the nasal cavities from a nostril or into the throat from the mouth.

It is also an object of the invention to evaluate the possibility of administering different substances in discrete form into a human or animal body, by injection into a body cavity, for purposes of therapeutic treatment or vaccination for example, and in particular of administering substances having a systemic action, not directly linked to the treatment of conditions affecting the ENT area, by introduction and diffusion into the nasal cavities.

The device according to the invention can also be used to evaluate the consequences of inhaling pollutant or toxic products in discrete form.

According to the invention, the device comprises:

an anatomical portion of an observed part of a dead subject, comprising at least in part the body cavity or cavities, and comprising a natural orifice with which at least one of said body cavities communicates; this anatomical portion is obtained by at least one section through said observed part, passing through the body cavity or cavities, and is treated for its preservation, for example by a technique called “plastination”, which consists in extracting water and fat from the tissues of this anatomical portion and replacing them with at least one nonputrefying synthetic material, for example a thermoset resin, either rigid or flexible;

at least one sensor which can detect and/or quantify said product at at least one site or zone of the body cavity or cavities;

at least one closure piece placed against said anatomical portion in the area of each sectioned surface, in such a way as to close each body cavity or part of the body cavity, but to leave access to said natural orifice, and

if appropriate, an introduction device placed opposite said natural orifice, or engaged partially in this orifice, and able to introduce said product in accordance with predetermined parameters.

The term “introduction” is also intended to signify injection, spraying and atomizing, or any other method of diffusion of the product of interest, directly or indirectly, within the body cavity being studied.

The structure of certain body cavities, in particular the nasal cavities and sinuses, is in some cases very complex at the anatomical level and cannot in any way be reproduced faithfully by a synthetic model. By contrast, the technique of plastination used on a dead subject makes it possible to a very large extent to conserve the anatomical structure of these cavities, in particular the nasal cavities and the sinuses; moreover, this technique permits preservation of the mucous membranes, which appear to have a significant effect on the diffusion of a product in discrete form in a body cavity.

The technique of plastination used in the context of the invention consists in fixing tissues in a solution of formaldehyde, then extracting water in acetone at low temperature, to replace the water and fats contained in the tissues, then forced impregnation by submersion in a bath of nonputrefying synthetic material, for example a thermoset resin.

The product or the substance of interest can in particular be in the form of liquid droplets, solid or semi-solid particles, powder, vapor or microcapsules.

At least one sensor can be placed in a body cavity; it is in this case advantageously arranged so as to be flush with the surface of the wall in which it is placed, so as not to risk disturbing the flow of product in the cavity or cavities to be studied.

This sensor can be of any type suitable for detecting the presence of the product of interest. It can in particular be a humidity sensor, an optical sensor or an impact sensor.

The device can also comprise at least one sensor situated not in a body cavity but outside the latter, for example in the anatomical portion studied, this sensor being movable. This can be in particular a camera or an endoscope.

The aforementioned closure piece or pieces are advantageously removable, for example in order to permit cleaning or drying of the body cavity or cavities between two injections of product.

This piece or these pieces can advantageously be transparent in order to make it possible to visualize the diffusion of the product during and after its introduction and to detect any leaks.

At least one of these pieces can be opaque to radiation of types α, β, β, UV or IR, for example for protecting the person using the device.

The device preferably comprises an introduction device and adjustable means for positioning said device, making it possible to set the position and/or orientation of this device relative to said anatomical portion.

According to a preferred application of the invention, said anatomical portion is a portion of the human head. Said body cavity or cavities are then preferably one or more nasal cavities and/or sinuses. The device according to the invention is thus intended in particular for characterizing the diffusion of a product in discrete form, in particular an aerosol, in a nasal cavity and/or sinus of a subject when this product is injected from a nostril.

The head can be sectioned on a median anteroposterior plane, passing immediately behind the nasal septum. This nasal septum is thus eliminated so as not to impede the placement of the sensors.

The head can also be sectioned on an anteroposterior plane passing through the area of the maxillary sinus, for example substantially in the plane passing through the pupil.

In order to obtain the anatomical portion being studied, the head can be sectioned on any sectional planes appropriate for studying the cavity which is being observed, for example auditory system, sinuses, etc.

The head is preferably sectioned on these two planes, for example median anteroposterior plane and anteroposterior plane, respectively, in order to characterize the diffusion of the product of interest simultaneously in a nasal cavity and in the maxillary sinus.

By adjusting the introduction device it is possible to achieve introduction and/or diffusion results in various positions or orientations of said device in relation to the anatomical portion, or in any portion or orientation in relation to any two-dimensional or three-dimensional reference system.

In this case, the adjustable positioning means are advantageously equipped with at least one graduation and at least one marker making it possible to set the position and/or orientation of the introduction device.

The device according to the invention can also comprise a unit for processing the signals which are emitted by the sensor or sensors, able to deliver a signal or signals representing the presence and/or concentration of product in discrete form in the area of said sensor or sensors.

The device according to the invention can additionally comprise means which are able to generate a movement of fluid (liquid or gaseous) in said body cavity or cavities.

These means make it possible to recreate a respiration or inhalation flow. They can in particular involve an anesthesia ventilator.

To ensure that it is clearly understood, the invention will be further described below with reference to the attached diagrammatic drawing which shows, by way of nonlimiting example, a preferred embodiment of the device to which it relates.

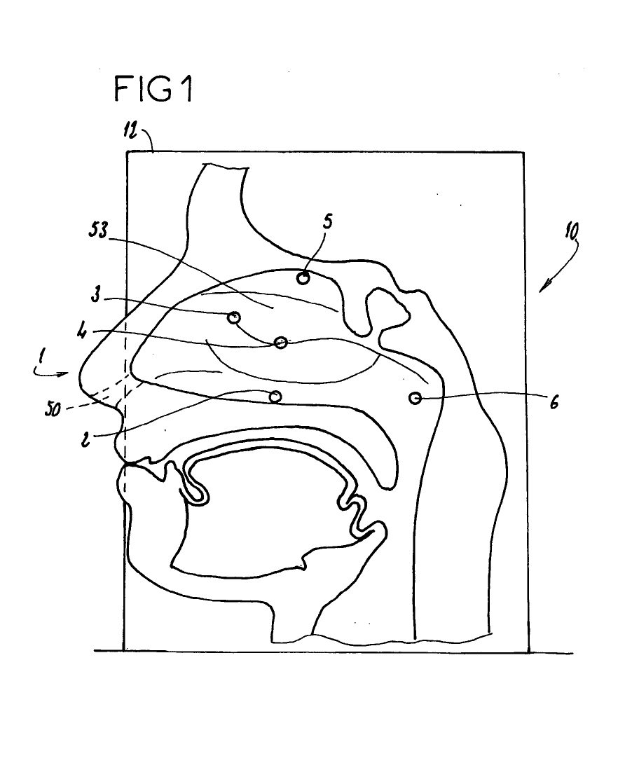

FIG. 1 is a view of a portion of the human head treated by “plastination” and sectioned in particular on a substantially median anteroposterior plane; and

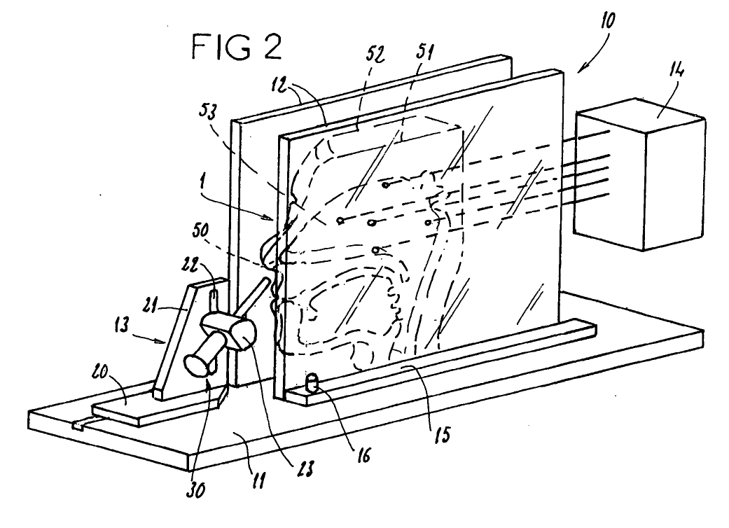

FIG. 2 shows a perspective view of the device according to the invention.

FIG. 1 shows a portion of the human head 1 sectioned on a median anteroposterior plane 51 and on a plane 52 passing substantially through the median zone of the maxillary sinus. The median section has been performed immediately behind the nasal septum so as to eliminate this septum but retain the maximum volume of the nasal cavity.

The portion 1 has been obtained from the head of a dead subject and has been treated to permit its preservation, by a technique called “plastination”. This technique is for example as follows:

1) Removal of a head less than forty-eight hours after the death of the subject; the period elapsing between the time of death and the removal does in fact have a direct bearing on the quality of the portion obtained, as the nasal mucosa is very fragile, especially in the area of the conchae, which have important vasoactive qualities.

2) Fixing of the head in a 10% formaldehyde solution for at least four weeks and preferably for three months, at a temperature of the order of 5° C.

3) Anatomical preparation (especially removal of the brain), and carrying out the aforementioned sections.

4) Submersion of the obtained portion 1 in acetone at −25° C. for a period sufficient to ensure that the acetone replaces the water and lipids in the tissues by freeze substitution. This period of time is generally of the order of four weeks.

5) Submersion of the portion 1 in a bath of fluid silicone at a temperature of −25° C. and for a period of from twenty-four to forty-eight hours. The silicone is for example “S10” which is sold commercially under the tradename “BIODUR”.

6) Gradual application, while portion 1 is still submerged, of an atmospheric vacuum for ten to twenty days, so as to extract the acetone from the tissues and induce the silicone to enter these tissues.

7) Draining of the silicone for forty-eight hours and polymerization of this silicone.

Dichloromethane can be used in place of acetone.

Epoxy resins can be used in place of silicone.

In the example shown, five humidity sensors of the capacitive type have been placed on the portion 1, namely (cf. FIG. 2):

a sensor 2 in the area of the horizontal plate of the palatine bone,

a sensor 3 in the area of the middle concha,

a sensor 4 in the area of the median meatus,

a sensor 5 in the area of the cribriform plate, and

a sensor 6 in the area of the cavum.

These sensors 2 to 6 can in particular be those available under the tradename “LJK technologie®”.

FIG. 2 shows a device 10 making it possible, with the aid of the portion 1 equipped with sensors 2 to 6, to characterize the diffusion of a product in discrete form, in particular an aerosol, in the nasal cavity and/or the maxillary sinus of the portion 1.

As is shown, the device 10 comprises a lower plate 11, or base, two parallel plates 12 perpendicular to the plate 11 and receiving between them the head portion 1, equipped with the sensors 2 to 6, means 13 for supporting and positioning an introduction device 30, and a unit 14 for processing the signals transmitted by the sensors 2 to 6.

The plate 11 comprises threaded holes for rules 15 used to position the plates 12 by means of screws 16.

The plates 12 are for example made of a transparent material of the “plexiglass” type. They are applied tightly to the surfaces of the portion 1 which result from the aforementioned sections and make it possible to enclose the nasal cavity 53 and the maxillary sinus of this portion 1. A seal can be placed between the portion 1 and the plates 12.

The means 13 comprise a base 20 fixed by screws to the plate 11, a wing 21 in which a slot 22 is formed, and a support 23, the latter including the introduction device 30.

The support 23 bears against the wing 21 and has a threaded hole in which it receives a screw which is engaged sliding in the slot 22, this screw bearing with its head against the wing 21 on the side remote from the support 23. This screw can be loosened to adjust the height and inclination of the introduction device 30 in a vertical plane.

The wing 21 and/or the support 23 can be equipped with a graduation and markers with which it is possible, for example, to set said height and/or said inclination on the basis of precise values.

The sensors 2 to 6 are connected to the processing unit 14. The latter processes the signals emitted by these sensors and delivers to an electronic unit (not shown) signals representative of the presence and/or concentration, in the area of said sensors 2 to 6, of the product of interest which has been introduced and diffused.

As will be apparent from what has been stated above, the invention makes available a device for reliably and precisely characterizing the diffusion of a product in discrete form in one or more body cavities, in particular a nasal cavity and/or a sinus.

It goes without saying that the invention is not limited to the embodiment described above by way of example and that instead it encompasses all the alternatives thereof coming within the scope of protection defined by the attached patent claims.

Other body cavities, such as the lungs, throat, stomach, can be studied and characterized using any device constructed or obtained according to the present invention.

1. A device for characterizing the diffusion of a product of interest in discrete form in one or more body cavities (53) at least, in particular a nasal cavity and/or a sinus, wherein said device comprises:
an anatomical portion (1) of an observed part of a dead subject, comprising at least in part the body cavity or cavities (53), and comprising a natural orifice (50) with which at least one of said body cavities communicates; this anatomical portion (1) is obtained by at least one section (52, 51) through said observed part, passing through the body cavity or cavities, and is treated for its preservation, for example by a technique called “plastination”, which consists in extracting water and fat from the tissues of this anatomical portion (1) and replacing them with at least one nonputrefying synthetic material, for example a thermoset resin;
at least one sensor (2 to 6) which can detect and/or quantify said product at at least one site or zone of the body cavity or cavities;
at least one closure piece (12) placed against said anatomical portion (1) in the area of each sectioned surface, in such a way as to close each body cavity (53) or part of the body cavity, but to leave access to said natural orifice (50), and
if appropriate, an introduction device (30) placed opposite said natural orifice, or engaged partially in this orifice, and able to introduce said product in accordance with predetermined parameters.

2. The device as claimed in claim 1, wherein at least one sensor (2 to 6) is placed in a body cavity (53) and is arranged so as to be flush with the surface of the wall in which it is placed.

3. The device as claimed in claim 2, wherein said device comprises at least one sensor (2 to 6) situated outside a body cavity, for example in the anatomical portion, this sensor being movable.

4. The device as claimed in claim 1, wherein the closure piece or pieces (12) are removable.

5. The device as claimed in one of claims 1 through 4, wherein the closure piece or pieces (12) are transparent.

6. The device as claimed in one of claims 1 through 5, wherein said anatomical portion (1) is a portion of the human head.

7. The device as claimed in claim 6, wherein said body cavity or cavities (53) are one or more nasal cavities and/or sinuses.

8. The device as claimed in claim 7, wherein said head (1) is sectioned on a median anteroposterior plane, (51) passing immediately behind the nasal septum.

9. The device as claimed in claim 7 or claim 8, wherein said head (1) is sectioned on an anteroposterior plane (52) passing through the area of the maxillary sinus, for example substantially in the plane passing through the pupil.

10. The device as claimed in one of claims 1 through 9, wherein said device comprises an introduction device (30) for the product of interest, and adjustable means (13) for positioning said device, making it possible to set the position and/or orientation of this device relative to said anatomical portion (1).

11. The device as claimed in claim 10, wherein the adjustable positioning means (13) are equipped with at least one graduation and at least one marker making it possible to set the position and/or orientation of the introduction device (30).

12. The device as claimed in one of claims 1 through 11, wherein said device comprises a unit (14) for processing the signals which are emitted by the sensor or sensors (2 to 6), able to deliver a signal or signals representing the presence and/or concentration of product in the area of said sensor or sensors (2 to 6).

13. The device as claimed in one of claims 1 through 12, wherein said device comprises means which are able to generate a movement of fluid (liquid or gaseous) in said body cavity or cavities.

14. The device as claimed in claim 12 or claim 13, wherein said device comprises a lower plate (11), two plates (12) receiving between them the anatomical portion (1) equipped with sensors (2 to 6), said adjustable means (13) for positioning the introduction device (30), and said unit (14) for processing the signals transmitted by the sensors (2 to 6).
